(19) United States
(12) Patent Application Publication
(10) Pub. No.: US 2006/0057067 A1
(43) Pub. Date: Mar. 16, 2006

(54) Lymph-like composition and method to prevent and treat central nervous system injuries

(76) Inventor: Yanming Wang, Malden, MA (US)

Correspondence Address: YANMING WANG, 203 SUMMER STREET, MALDEN, MA, 02148, US

(21) Appl. No.: 11/193,181

(22) Filed: Jul. 28, 2005

Related U.S. Application Data
(63) Continuation of application No. 10/939,253, filed on Sep. 11, 2004.

(51) Int. Cl. A61K 38/28; A61K 31/7076; A61K 49/00; A61K 33/06

(52) U.S. Cl. 424/9.1; 514/3; 514/47; 424/682

(57) ABSTRACT

The existence of the cerebrospinal fluid and the intracranial pressure contribute the susceptibility of the CNS to injuries. A lymph-like composition and method for treating brain and spinal cord injuries are provided. The lymph-like composition comprises Polypeptides, Insulin, Mg⁺ and ATP in an artificial cerebrospinal fluid. The method includes: a). Administering an agent to reduce the CSF production, b). Withdrawing a volume of cerebrospinal fluid, and c). Repeatedly injecting and withdrawing an effective amount of lymph-like composition through the subarachnoid space to wash the central nervous system tissue where protection is needed, and finally removing an effective amount of lymph-like composition to maintain a lower the intracranial pressure.

This is a continuation of the patent application filed Sep. 11, 2004, Ser. No. 10/939,253.

### BACKGROUND OF THE INVENTION

1. Field of the Invention

This invention is related to a lymph-like formulation and a method of using the formulation to prevent and treat the brain and spinal cord injuries in patients.

2. Background Information

The central nervous system (CNS), which consists of the brain and spinal cord, is very susceptible to injuries, as compared with other organs such as lung, liver, kidney, and intestines. The mechanism(s) underlying this susceptibility are not completely understood. Many theories have been proposed and intensively investigated. Factors considered to be causative in neuronal injury include oxygen free radicals, calcium overloading, excitatory amino acid release, and nitric oxide. However, the search for a neuroprotective treatment based on these various molecular mechanisms has yielded disappointing results during clinical trials, and therefore, these various pathways have not been convincingly demonstrated to be key factors that are responsible for the vulnerability of the brain and spinal cord to injuries.

Incidents that tend to cause CNS injuries, such as hypoxia-ischemia, trauma, infection, poisoning, and cardiac arrest, are invariably accompanied by rapid development of cerebral edema. Cerebral edema refers to the water retention in both interstitial space and inside cells of the CNS, which leads to increased intracranial pressure (ICP). The increase in ICP can cause brain damage directly through mechanical force and indirectly through a “secondary” blood perfusion deficit caused by the collapse of blood vessels.

Much effort has been made in studying the causative factors of brain edema. Most researches focus on water loss from blood vessels, but seldom pay any attention to the cerebrospinal fluid (CSF), the watery environment that bathes the entire brain and spinal cord. As the direct source of water in neural edema, CSF should be given a more important role in the study of neuroprotection.

The CNS lacks a lymphatic system, the fluid environment surrounding organs outside the CNS such as liver and lungs. The CSF system, however, is very different from the lymph system.

Lymph has almost the same composition as interstitial fluid and plasma.

The protein concentration in the interstitial fluid and lymph of most tissues averages about 2-6 gm/dl. An important function of the lymphatic system is to serve as a “scavenger” system that removes excess fluid from the interstitium, i.e., spaces between cells. Blockage of lymphatic flow is known to result in severe clinical edema. The interstitial fluid pressure outside the CNS is believed to be negative, which is largely because of the lymphatic system.

Although, in the past, some researchers regarded the CSF as the lymphatic system of the CNS, the CSF system is actually very different in several aspects from the lymphatic system. Two of the differences between the CSF and lymphatic systems prove crucial in the development of neural edema.

First, unlike the lymph or plasma, the CSF contains much lower concentrations of proteins, which are strong water binding molecules. The CSF is secreted from plasma through choroid plexuses that line the cerebral ventricles. Tight junctions linking the adjacent choroidal epithelium and forming what is known as the Blood-CSF Barrier prevents most large molecules from effectively passing into the CSF from the blood. Many large molecules are polypeptides and are very important for cell survival.

Second, unlike the lymphatic system that has a negative interstitial pressure, the CNS has a positive interstitial fluid pressure.

It is demonstrated through this invention that the low protein concentration and positive interstitial fluid pressure make the CNS prone to rapid water retention (or edema) after the initial insult. The treatment disclosed in this invention are based on mimicking the lymphatic system in these two aspects by the following measures: (1) reducing interstitial pressure, and (2) increasing the concentration of water-binding polyaminio acids in the CSF.

Lowering the ICP reduces the interstitial pressure of CNS. Although it is beneficial, reducing ICP alone is not enough to reach the maximum neuroprotective effect. For example, the CSF drainage has been used to prevent spinal cord damage caused by cross-clamping aorta during aortic surgery for more than 50 years. The clinical outcome of this approach, however, has been inconsistent at best. This inconsistent result is likely caused by the CSF remained in the folds and chambers of the CNS after general CSF removal. The brain and spinal cord have complex contours with many sulci, gyri and pools. These complicated structures make it impossible to remove the CSF completely even when ICP is reduced to 0 mm Hg. Moreover, surface tension and capillary forces retain CSF in the Virchow-Robin space and in the spaces between the dura and brain surface. This invention addresses problem of treating the remaining CSF after general CSF removal.

Researchers have suggested that bolus infusion of hyperoncotic solution into the cerebral vasculature or perfusion of hyperoncotic artificial CSF can alleviate cerebral edema. The term “hyperonconic” refers to high colloid osmotic pressure caused by the existence of large molecular weight substances that do not pass readily across capillary walls. For example, U.S. Pat. No. 6,500,809 to Frazer Glenn discloses a method of treating neural tissue edema using hyperoncotic artificial CSF. Several colloid osmotic agents including albumin and dextran were used in the method.

This invention, however, reveals that the colloid osmotic pressure is not a key factor. Although albumin is effective in protecting the CNS tissue, it appears that its colloid osmotic effect is not the primary reason for its neural protective effect, because other colloid osmotic agents such as Dextran and Hetastarch are ineffective. In contrast, gelatins, even with molecular weights smaller than cut-off size for colloid osmotic agents are effective. In fact, gelatins with various molecular weights ranging from 20,000 to 100,000 Daltons are all effective regardless of their molecular weights. Collagen and Sericin are also effective. Albumin, gelatin, collagen, and Sericin all belong to poly amino acids category. It is thus the water-binding properties of proteins or other polyaminoacids that really matter.

The CNS can be made as resistant to various insults as other organ systems, or at least less vulnerable to such insults, by mimicking lymphatic system of other organs. The present invention is also directed at other mechanisms of ischemic injury that are common to all organ systems, including the use of insulin, magnesium and ATP.

The CSF contains about two third of plasma glucose concentration (CSF: 61 mg/dl; Plasma: 92 mg/dl). However it contains about at most one fifteenth of plasma insulin concentration (CSF: 0-4 μU/ml; fasting plasma: 20-30 μU/ml). Insulin is a polypeptide, with a molecular weight of about 6000 Daltons. Similar to albumin, it cannot easily enter the CSF through the blood-CSF barrier. Insulin has also been regarded as a growth factor, evidences have repeatedly proven that insulin yield protection for ischemic cerebral tissue independent of its glucose lowering effect. Compared with other growth factors, insulin has been used in clinic for years, and is much less expensive.

Magnesium (Mg²⁺) is the second highest electrolyte intracellularly (58 mEq/L). ATP (Adenosine 5′-triphosphate) is always present as a magnesium: ATP complex. Mg²⁺ basically provides stability to ATP. At least more than 260 to 300 enzymes have been found to require Mg²⁺ for activation. Best known among these are the enzymes involved in phosphorylations and dephosphorylations: AT-Pases, phosphatases, and kinases for glycolytic pathway and krebs cycles. At the level of the cell membrane Mg²⁺ is needed for cytoskeletal integrity, the insertion of protein into membranes, the maintenance of bilayer fluidity, binding of intracellular messengers to the membrane, regulation of intracellular Ca²⁺ release by inositol triphosphate etc. Mg²⁺ also affects the activities of pumps and channels regulating ion traffic across the cell membrane. The potential changes in tissue Mg²⁺ might also affect the tissue ATP levels. In tissue culture and animal models elevated Mg²⁺ concentration has been repeatedly proven to protect neurons and other cells.

The concentration of ATP inside cells is high, whereas the concentration outside cells is very low. Harkness and coworkers showed that the ATP concentrations is about 1 to 20 μmol/l in plasma, however in CSF, ATP could not be detected, and it was estimated to be about less than 0.05 μmol/l. Muñoz and co-workers detected that the ATP concentration in CSF is about 16 nM/l. Exogenous ATP provides direct energy to the damaged tissue. Sakama and coworkers showed that continuous application of ATP (100 μM) significantly increased axonal transport of membrane-bound organelles in anterograde and retrograde directions in cultured neurons. Uridine 5′-triphosphate produced an effect similar to ATP. Mg-ATP has been used clinically in Japan to treat hepatic and kidney hypoxia-ischemia.

Acidosis is a universal response of tissue to ischemia. In the brain, severe acidosis has been linked to worsening of cerebral infarction. Recent evidence however suggests that mild extracellular acidosis protects the brain probably through preventing activation of NMDA receptors and inhibition of Na⁺/H⁺ exchange. It has been reported mild acidosis provide cell protection down to pH 6.2. The acidosis that accompanies ischemia is an important endogenous protective mechanism. Correction of acidosis seems to trigger the injury. It has also been speculated that mild acidosis might stimulate anaerobic glycolysis that might supplement NADH oxidation and ATP yields.

Recombinant tissue plasminogen activator (rt-PA), a thrombolytic agent, has been shown to be effective to treat ischemic stroke if used within 3 hours after the onset.

### DETAILED DESCRIPTION OF ILLUSTRATIVE EMBODIMENTS

One of the embodiments aims to reduce interstitial fluid pressure in CNS, by administering an agent to inhibit the CSF production. Examples of this kind of treatment agents include Acetazolamide and Furosemide. Another embodiments is also based on reducing interstitial pressure in CNS, by removing the CSF from subarachnoid space to reduce the ICP.

Other embodiments of the present invention introduce a lymph-like composition during the critical period when CNS protection is need. The example components of the lymph-like composition include (1) Molecules primarily consisting of chemically-linked amino acids, (2) ionic magnesium (Mg²⁺), (3) adenosine triphosphate (ATP), and (4) insulin.

### The Polypeptides

Acting as his own lexicographer, the patentee calls the molecules that mainly consist of chemically-linked amino acids as “the Polypeptides” for the sake of simplicity. The Polypeptides have significant water-binding capacity. They include a wide variety of molecules, from small peptides containing two or more amino acids to proteins of large molecular weight and multiple peptide chains. The Polypeptides can be natural or synthetic molecules. They also include molecules that consist of amino acids and other building blocks such as hyaluronic acid or glucose (e.g., proteoglycan).

Whether the Polypeptides can pass through the capillary walls to generate colloid osmotic pressure are not important in this invention. In fact, colloid osmotic agents without the Polypeptides, such as Dextran, do not confer neuroprotective effect. It is preferred that the Polypeptides do not readily pass through cell membrane. Therefore, a molecular weight above 1000 Doltons is preferred. However, large molecules also have their disadvantage for being harder to be absorbed in subarachnoid space after the treatment. Therefore, the invention prefers, but is not limited to, Polypeptides with molecular weight between 5,000 to 30,000 Daltons.

Several examples the Polypeptides are described here, including albumin, collagen, and gelatin. Albumin is blood protein and an expensive option for the treatment, considering the current cost of albumin use already accounts for 10 to 30% of pharmacy budgets in hospital units.

Gelatins, on the other hand, can be a much cheaper option for the Polypeptide. Injectable gelatin polypeptides are much cheaper than albumin, and has been used in clinic in many countries such as Europe, China and South Africa. Examples of available commercial pharmaceutical gelatins include GELOFUSINE® and HAEMACCEL®. Heat shock protein can also be used for the Polypeptide. Example concentrations of the Polypeptides are ranged from 0.1-30 gram/dl. The preferred concentration range is between 1 and 10 gram per dl.

### Insulin, ATP, and Other Constituents

The insulin concentration should be in a range from 0.01 to 1000 μU/ml. The preferred insulin concentration is between 1 and 60 μU/ml. All growth factors having insulin-like effect can be chosen to replace insulin. For examples, insulin-like growth factors, nerve growth factor, brain derived neurotrophic factor, neurotrophin, fibroblast growth factor and glial cell line derived neurotrophic factor, erythroproietin, growth hormone, and growth hormone releasing factor may be used to replace insulin or may be used in combination with insulin.

The ATP concentration should be in a range from 16 nM to 5 mM. The preferred ATP concentration is between 0.001 to 1 mM. The most preferred ATP concentration is between 0.001 and 0.01 mM. Other high energy compound such as Uridine 5′-triphosphate can be used to replace ATP. The components and concentration range of the Mg²⁺ and artificial CSF can be as follow: Na 120-155 meq/L, K 0.1-5.0 meq/L, Ca 0.1-3.0 meq/L, P 0.1-2 meq/L, Cl 120-155 meq/L, Mg 0.4-8 meq/L, HCO₃ 0-25 meq/L, Glucose 0-60 mg/dl and water. The preferred concentration range of the Mg²⁺ and artificial CSF is as follow: Na 150 meq/L, K 3.0 meq/L, Ca 1.4 meq/L, P 1.0 meq/L, Cl 155 meq/L, Mg 2.5-5 meq/L, and water.

Normal blood pH value is about 7.35 to 7.45. The pH value of the composition should be in a range between 6.2 to 7.35. The pH value between 6.8-7.0 is preferred. The final osmolality should be between 280-340 mOsm/L.

To make the composition, molecules consisting of the Polypeptides, insulin, ATP and artificial CSF may be manufactured in a ready to use condition. Optionally, artificial CSF with elevated Mg²⁺ concentration may be manufactured in one container, the mixture of molecules consisting of the Polypeptides, insulin and ATP may be assembled in another container.

The composition may also contain other nutrients such as vitamins (e.g., D-Calcium Pantothenate, Choline, Folic acid, i-Inositol, Niacinamide, Pyridoxal, Riboflavin, Thiamine, Vitamin B₁₂ etc.), Amino acids (e.g., L-Alanine, L-Arginine, L-Asparagine, L-Cysteine, L-glutamine, L-glutamate, Glycine, L-Histidine, L-Isoleucine, L-leucine, L-lysine, L-methionine, L-Phenylalanine, L-proline, L-serine, L-threonine, L-tryptophan, L-tyrosine, L-valine etc.), phospholipids, Cholesterol, fat, fatty acid, D,-L-alpha-tocopherol, antioxidant etc.

The lymph-like or a plasma-like composition may also contain oxygen carriers such as bis-perfluorobutyl ethylene (oxygenated before use), intermediate molecules of glycolysis (e.g., fructose-1,6-biphophate, glyceraldehyde-3-phosphate, 1,3 bisphosphoglycerate, 3-phosphoglycerate, 2-phosphoglycerateare, phosphoenolpyruvate, pyruvate), enzymes for glycolysis (e.g., hexokinase, phosphoglucose isomerase, phosphofructokinase, aldolase, triosephosphate isomerase, glyceraldehydes 3-phosphate dehydrogenase, phosphoglygerate kinase, pyruvate kinase etc.), fructose-2,6-biphosphate, and intermediates of Krebs cycle.

The lymph-like or a plasma-like compositions herein may also be advantageously combined with any of the agents used to treat stroke or other neurological deficiencies based on other mechanisms including: calcium channel blockers such as Nimodipine and Flunarizine; calcium chelators such as DP-b99, potassium channel blockers, Free radical scavengers (e.g., antioxidants such as Ebselen, porphyrin catalytic antioxidant manganese (III) meso-tetrakis (N-ethylpyridinium-2-yl) porphyrin, (MnTE-2-PyP (5+)), disodium 4-[(tert-butylimino) methyl] benzene-1,3-disulfonate N-oxide (NXY-059), N:-t-butyl-phenylnitrone or Tirilazad), GABA agonists including Clomethiazole, GABA receptor antagonists, glutamate antagonists (e.g., AMPA antagonists such as GYKI 52466, NBQX, YM90K, YN872, ZK-200775 MPQX, Kainate antagonist SYM 2081, NMDA antagonists such as CGS 19755, NMDA channel blockers including Aptiganel (Cerestat) and CP-101,606, Dextrorphan, destromethorphan, magnesium, metamine, MK-801, NPS 1506, and Remacemide), glycine site antagonists including ACEA 1021 and GV 150026, polyamine site antagonists such as Eliprodil, and Ifenprodil, adenosine receptor antagonists, Nitric oxide inhibitors including Lubeluzole, opiod antagonists such as Naloxone and Nalmefenem, Phosphatidylcholine precursor, Citicoline (CDP-coline), serotonin agonists including Bay x 3072, Sodium channel blockers (e.g., Fosphenyloin, Lubeluzole, and 619C89), potassium channel openers such as BMS-204352, anti-inflamatory agents, protein kinase inhibitors, and other active agents that provide energy to cells such as co-enzyme A, co-enzyme Q, or cytochrome C. Similarly, agents known to reduce cellular demand for energy, such as phenyloin, barbital, or lithium may also be added. These agents may be added into this lymph-like composition or may be administered orally or intravenously in combination with this invented composition and method.

The invention also includes methods of treating or preventing CNS injuries. The preferred embodiments include three approaches that can be used separately or in combination. First, administer a substance or composition in an amount sufficient to reduce the CSF production. The administration can be by oral, intravenous, intramuscular, or intrathecal route. For examples, Furosemide 40-60 mg or Acetazolamide 0.25-0.5 g can be injected intravenously.

Second, remove CSF from the patient's subarachnoid space. For a localized CNS injury, such as stroke, spinal cord trauma, the CSF may be removed from one puncture point in lumbar subarachnoid space or in cisterna magna or in subarachnoid space of direct affected area. For more general CNS injury, such as cardiac arrest, severe head trauma and CNS protection during cardiac or aortic bypass surgery etc., the CSF may be removed from more puncture points, and may even from the lateral cerebral ventricles. The CSF removal reduces the ICP and cuts off the major water supply to CNS tissue where protection is needed. By removing the CSF, the ICP can be reduced even to 0 mm Hg if necessary. For a localized CNS injury, less amount of CSF may be removed. For maximums CNS protection, the CSF should be removed as completely as possible to create a “CSF free environment” around the CNS tissue where protection is needed. To maximize the amount of the CSF removed, the patient's body position may be adjusted, allowing that the drainage point was at the lowest level, using gravity to facilitate the removal of the CSF, for example, a patient may maintain a sitting position while the CSF is being removed from lumbar subarachnoid space.

Third, introduce a lymph-like composition described above. The aim of this step is to treat any remaining CSF following general manual CSF removal, although it may be used independently by itself. As discussed above, the remaining CSF in sulci, gyri, pools, and particularly in the Virchow-Robin space is still harmful to injured CNS tissue. By introducing the lymph-like composition into the subarachnoid space around injured CNS, the remaining CSF of the inaccessible spaces will be diluted and finally replaced by the lymph-like composition. After removal of the CSF, the lymph-like composition will be injected into the subarachnoid space through the puncture point where the CSF was removed. The injected lymph-like composition is approximately equal or less to the amount of CSF removed. The injected lymph-like composition may be withdrawn then injected back repeatedly for several times to “wash” the CNS tissue where the protection is needed. This “wash” procedure may be performed through one or more puncture points, injecting at one point while withdrawing at other point(s). The “wash” procedure may take from one minute to a few ten minutes, or may take hours in complicated case. The lymph-like composition may or may not be re-used for the “wash” procedure. Finally, a mount of the lymph-like composition will be removed to reduce the ICP after the “wash” procedure. The ICP may be maintained at range between 0 and 15 mm Hg with lymph-like composition. The lower the ICP is, the better the outcome. It is preferred that the final ICP is maintained at 0-7 mm Hg. The CSF in sulci, gyri, pools, and the Virchow-Robin space is diluted and replaced by the lymph-like composition nourishing the injured CNS. The “wash” procedure can be repeated every 3-4 hours or as needed. Optionally, the lymph-like composition may be replaced by blood plasma or serum during the “wash” procedure.

Alternatively, patient's own CSF may be used to replace artificial CSF in making the lymph-like composition. Usually 5-160 ml of the patient's own CSF can be obtained as a solvent to dissolve the mixture of molecules consisting of the Polypeptides, insulin and Mg⁺-ATP. Elliot B solution is an artificial CSF that has been approved as a solvent since 1996 in USA. Elliot B solution may also be used to replace artificial CSF.

The present invention can be combined with thrombolytic agents such as recombinant tissue plasminogen activator (rtpA), streptokinase, and tenecteplase in dissolving thrombosis in management of stroke.

### EXAMPLE ONE

Making of a Lymph-Like Composition for Protecting CNS Tissue

Artificial CSF with higher concentration of Mg⁺ used in this example is made according to table 1.

TABLE 1

| Components | Amount |
|---|---|
| NaCl | 8.182 gram |
| KCl | 0.224 gram |
| CaCl₂.2H₂O | 0.206 gram |
| Na₂HPO₄ | 0.113 gram |
| NaH₂PO₄ | 0.023 gram |
| MgSO₄ | 0.361 gram |
| Glucose | 0.6 gram |

Sterile water for dilution to 1000 ml

Mixture of Albumin (molecular weight 68,000 Daltons), Insulin and ATP used in this example is made according to table 2.

TABLE 2

| | | |
|---|---|---|
| Albumin | 80 | gram |
| Insulin | 3,000 | μU |
| ATP | 0.55 | milligram |

Mix these substances in one container

To make the composition, dissolve the mixture of Albumin, Insulin and ATP in artificial CSF. Final pH of the composition is adjusted between 6.8 to 7.0.

### EXAMPLE TWO

Making of a Lymph-Like Composition for Protecting CNS Tissue

Artificial CSF with higher concentration of Mg⁺ used in this example is made according to table 3.

TABLE 3

| Components | Amount |
|---|---|
| NaCl | 8.182 gram |
| KCl | 0.224 gram |
| CaCl₂.2H₂O | 0.206 gram |
| Na₂HPO₄ | 0.113 gram |
| NaH₂PO₄ | 0.023 gram |
| MgSO₄ | 0.361 gram |

Sterile water for dilution to 1000 ml

Mixture of Gelatin (molecular weight between 20,000-25,000 Daltons), Insulin and ATP used in this example is made according to table 4.

TABLE 4

| | | |
|---|---|---|
| Gelatin | 35 | gram |
| Insulin | 3,000 | μU |
| ATP | 0.55 | milligram |

Mix these substances in one container

To make the composition, dissolve the mixture of Gelatin, Insulin and ATP in artificial CSF. Final pH of the composition is adjusted between 6.8 to 7.0.

### EXAMPLE THREE

Treatment for Brain Ischemia

The focal cerebral ischemia was induced in 40 rats weighing between 200-250 gram. Group one (10 rats): control treatment with artificial CSF while maintaining ICP at 10 mm Hg. Group two (10 rats): treatment with the composition made according to example one while maintaining ICP at 0 mm Hg. Group three (10 rats): treatment with the composition made according to example one while maintaining ICP at 10 mm Hg. Group four (10 rats): treatment with the composition made according to example two while maintaining ICP at 0 mm Hg.

Ketamine/xylazine 30 mg/kg ip was given for anesthesia. A silicone catheter (0.025 OD, 0.012 ID inch) was surgically implanted in the cisterna magna as a route for removing the CSF and monitoring ICP. A hole of 3 mm in diameter was drilled on the left side of skull (3 mm lateral to midline and 3 mm in front of the bregma), dura was punctured, another silicone catheter (0.025 OD, 0.012 ID inch) was placed into the subarachnoid spaces on the surface of the forebrain.

Focal cerebral ischemia: A midline incision on the neck was made. The left common carotid artery, the external carotid artery (ECA) and the internal carotid artery (ICA) were exposed. The ECA was ligated and severed. A 3.0 nylon suture was advanced from the ECA to ICA to block the origin of left middle cerebral artery. The nylon suture was left in place for 24 hours to produce permanent focal cerebral ischemia on left hemisphere supplied by middle cerebral artery.

In group one, at 15 min after focal brain ischemia, rats received furosemide at 40 mg/kg im, followed by infusing 3 ml of artificial CSF (Table 5) through the catheter in subarachnoid spaces on the surface of the forebrain. The ICP was maintained at 10 mm Hg by infusing and withdrawing the artificial CSF.

TABLE 5

| Component | Amount |
|---|---|
| NaCl | 8.66 gram |
| KCl | 0.224 gram |
| CaCl₂.2H₂O | 0.206 gram |
| Na₂HPO₄ | 0.113 gram |
| NaH₂PO₄ | 0.023 gram |
| MgSO₄ | 0.096 gram |
| Glucose | 0.6 gram |

Sterile water for dilution to 1000 ml, pH adjusted to 6.8-7.4

In group two, at 15 min after focal brain ischemia, rats received furosemide at 40 mg/kg im. Then the CSF was removed as completely as possible from the silicone catheter in the cisterna magna and the subarachnoid space on the surface of the forebrain. After the CSF removal, 3 ml of the lymph-like composition made according to example one (consisting of albumin, insulin, ATP and artificial CSF of higher Mg⁺ concentration) was used to “wash” the injured brain area by repeatedly injecting and withdrawing the composition from the catheter in subarachnoid spaces on the surface of the forebrain. After the “wash” procedure, the lymph-like composition was removed from the catheters in the cisterna magna and subarachnoid spaces on the surface of the forebrain, the ICP was maintained at 0 mm Hg.

In group three, at 15 min after focal brain ischemia, rats received furosemide at 40 mg/kg im. Then the CSF was removed as completely as possible from the silicone catheter in the cisterna magna and the subarachnoid space on the surface of the forebrain. After the CSF removal, 3 ml of the lymph-like composition made according to example one (consisting of albumin, insulin, ATP and artificial CSF of higher Mg⁺ concentration) was used to “wash” the injured brain area by repeatedly injecting and withdrawing the lymph-like composition from the catheter in subarachnoid spaces on the surface of the forebrain. After the “wash” procedure, the ICP was maintained at 10 mm Hg by injecting or withdrawing the lymph-like composition through the catheters in the cistema magna and subarachnoid spaces on the surface of the forebrain

In group four, at 15 min after focal brain ischemia, rats received furosemide at 40 mg/kg im. Then the CSF was removed as completely as possible from the silicone catheter in the cistema magna and the subarachnoid space on the surface of the forebrain. After the CSF removal, 3 ml of the lymph-like composition made according to example two (consisting of gelatin, insulin, ATP and artificial CSF of higher Mg⁺ concentration) was used to “wash” the injured brain area by repeatedly injecting and withdrawing the lymph-like composition from the catheter in subarachnoid spaces on the surface of the forebrain. After the “wash” procedure, the lymph-like composition was removed from the catheters in the cistema magna and subarachnoid spaces on the surface of the forebrain, the ICP was maintained at 0 mm Hg.

Neurological deficit study: At 24 hours after cerebral ischemia, all rats were evaluated for neurological deficit. A score of 0-4 was used to assess the motor and behavioral changes. Score 0: No apparent deficits. Score 1: Contralateral forelimb flexion. Score 2: Decreased grip of the contralateral forelimb while tail pulled. Score 3: Spontaneous movement in all directions; contralateral circling only if pulled by tail. Score 4: Spontaneous contralateral circling.

Infarct volume study: After behavioral test, all rats were euthanized. Brains were taken out. 2,3,5-triphenyltetrazolium chloride (TTC) staining was used to distinguish the viable tissue and necrotic tissue. Sections of 1 mm in thickness were cut and stained with 2% TTC in phosphate buffer at 37° C. for 10 minutes. The sections were fixed in 10% formalin. Percentage of infarct volumes were calculated and analyzed with a computer.

Results: in group one, the average neurological deficit score was 3.89±0.09, the average infarct volume was 46.13±3.68%. In group two, the average neurological deficit score was 0.99±0.64, the average infarct volume was 9.85±0.69%. In group three, the average neurological deficit score was 2.01±0.21, the average infarct volume was 18.81±0.61%. In group four, the average neurological deficit score was 0.98±0.65, the average infarct volume was 8.96±0.78%.

It was concluded that the composition made according to example one and two significantly protected ischemia challenged brain. (P<0.01, group one vs. group two, three or four). ICP reduction is also important in protecting brain (p<0.05 group three vs. group two or four). Maximums protective effect could be obtained by combined steps of: 1). Administering furosemide im. 2). Removing the CSF to reduce the ICP. 3). Introducing the lymph-like composition in subarachnoid space to wash the injured brain to replace any remaining CSF and finally removing the lymph-like composition to reduce the ICP.

The examples and embodiments described above are used for the purpose of explanation. They should not be construed as limitations to the scope of the invention.

1. A lymph-like composition for protecting the central nervous system of a mammal comprising an artificial cerebrospinal fluid and at least one component selected from the following: the Polypeptides (molecules primarily consisting of chemically-linked amino acids), insulin, ATP and elevated concentration of Mg⁺.

2. A lymph-like composition for protecting the central nervous system of a mammal, as claimed in claim 1, wherein said artificial cerebrospinal fluid comprises: Na 120-155 meq/L, K 0.1-5.0 meq/L, Ca 0.1-3.0 meq/L, P 0.1-2 meq/L, Cl 120-155 meq/L, Mg 0.4-8 meq/L, HCO₃ 0-25 meq/L, Glucose 0-60 mg/dl and water.

3. A lymph-like composition for protecting the central nervous system of a mammal comprising the Polypeptides (molecules primarily consisting of chemically-linked amino acids) in an artificial cerebrospinal fluid and at least one component selected from the following: insulin, ATP and elevated concentration of Mg⁺.

4. A lymph-like composition for protecting the central nervous system of a mammal, as claimed in claim 1, wherein said Polypeptides are gelatins.

5. A lymph-like composition for protecting the central nervous system of a mammal, as claimed in claim 1, wherein the concentration of said Polypeptides is about 0.1-30 gram per 100 ml.

6. A lymph-like composition for protecting the central nervous system of a mammal, as claimed in claim 1, wherein said insulin is present in a concentration of about 0.01-1000 μU/ml.

7. A lymph-like composition for protecting the central nervous system of a mammal, as claimed in claim 1, wherein said ATP is present in a concentration of about 0.001 mM to 1 mM.

8. A lymph-like composition for protecting the central nervous system of a mammal according to claim 1, further comprising at least one component in an effective amount selected from the following: Vitamins (such as, D-Calcium Pantothenate, Choline, Folic acid, i-Inositol, Niacinamide, Pyridoxal, Riboflavin, Thiamine, Vitamin B₁₂ etc.), Amino acids (such as, L-Alanine, L-Arginine, L-Asparagine, L-Cysteine, L-glutamine, L-glutamate, Glycine, L-Histidine, L-Isoleucine, L-leucine, L-lysine, L-methionine, L-Phenylalanine, L-proline, L-serine, L-threonine, L-tryptophan, L-tyrosine, L-valine etc.), phospholipids, Cholesterol, fat, fatty acid, growth factors (such as nerve growth factor, Fibroblast Growth Factor, brain derived neurotrophic factor, insulin like growth factor, neurotrophin, erythroproietin, growth hormones, growth hormone releasing factor), and other active agents that provide energy to cells (such as co-enzyme A, co-enzyme Q, or cytochrome C), agents known to reduce cellular demand for energy (such as phenyloin, barbital, or lithium). D,-L-alpha-tocopherol, intermediates of glycolysis (such as fructose-1,6-biphophate, glyceraldehyde-3-phosphate, 1,3 bisphosphoglycerate, 3-phosphoglycerate, 2-phosphoglycerateare, phosphoenolpyruvate, pyruvate, lactate), enzymes for glycolysis (such as hexokinase, phosphoglucose isomerase, phosphofructokinase, aldolase, trio-sephosphate isomerase, glyceraldehydes 3-phosphate dehydrogenase, phosphoglygerate kinase, pyruvate kinase etc.). intermediates of krebs cycle.

9. A lymph-like composition for protecting the central nervous system of a mammal according to claim 1, has pH value of about 6.8 to 7.0.

10. A lymph-like composition for protecting the central nervous system of a mammal, as claimed in claim 1, wherein said artificial cerebrospinal fluid comprises: Na⁺ 150 mEq/L, K⁺ 3.0 mEq/L, Mg²⁺ 2.51-5.9 mEq/L, Ca²⁺ 1.4 mEq/L, P 1.0 meq/L, Cl⁻ 155 mEq/L and water.

11. A lymph-like composition for protecting the central nervous system of a mammal, as claimed in claim 1, wherein said artificial cerebrospinal fluid is the cerebrospinal fluid withdrawn from the subject being treated.

12. A method for protecting the central nervous system of a mammal, comprising at least two steps selected from the followings:
a). Administering an agent to reduce the CSF production,
b). Withdrawing a volume of cerebrospinal fluid through at least one puncture point in subarachnoid space and cerebral ventricle, and
c). Repeatedly injecting and withdrawing an effective amount of said lymph-like composition according to claim 1 through at least one puncture point in subarachnoid space to wash the central nervous system tissue where protection is needed, and finally removing an effective amount of said lymph-like composition to maintain the intracranial pressure at less than 10 mm Hg.

13. A method for protecting the central nervous system of a mammal according to claim 12, in step a), wherein said agent to reduce the CSF production is Furosemide or Acetazolamide.

14. A method for protecting the central nervous system of a mammal according to claim 12, in step b), wherein said cerebrospinal fluid is withdrawn as completely as possible.

15. A method for protecting the central nervous system of a mammal, comprising at least two steps selected from:
a). Administering an agent to reduce the CSF production,
b). Withdrawing a volume of cerebrospinal fluid through at least one puncture point in subarachnoid space and cerebral ventricle, and
c). Repeatedly injecting and withdrawing an effective amount of said lymph-like composition according to claim 1 through at least one puncture point in subarachnoid space to wash the central nervous system tissue where protection is needed, and finally removing an effective amount of said lymph-like composition to maintain the intracranial pressure at normal ranges (10-15 mm Hg).

16. A method for treating ischemic stroke in a mammal requiring such treatment according to claim 12, comprising of added step of administering recombinant tissue plasminogen activator (rt-PA) to said mammal in an amount effective to restore blood flow to central nervous system tissue.

17. A method for treating ischemic stroke in a mammal requiring such treatment according to claim 12, in step c), wherein said lymph-like composition to wash the central nervous system tissue is blood serum or plasma derived from the subject being treated.

18. A lymph-like composition for protecting the central nervous system of a mammal according to claim 1, further comprises of at least one component in an effective amount selected from the following: calcium channel blockers, calcium chelators, oxygen carriers (such as bis-perfluorobutyl ethylene and oxygenated before use), Sodium channel blockers, potassium channel blockers, potassium channel openers, free radical scavengers—Antioxidants, GABA agonists, GABA receptor antagonists, polyamine site antagonists, Glycine site antagonists, protein kinase inhibitors, Serotonin agonists, Nitric oxide inhibitors, opiod antagonists, glutamate antagonists, AMPA antagonists, adenosine receptor antagonists, Kainate antagonist, NMDA antagonists (such as CGS 19755, Nimodipine, DP-b99 and Flunarizine, Aptiganel, CP-101,606, Dextrorphan, destromethorphan, metamine, MK-801, NPS 1506, GYKI 52466, NBQX, YM90K, YN872, ZK-200775, MPQX, SYM 2081, Bay x 3072, Remacemide, ACEA 1021, GV 150026, Clomethiazole, Eliprodil, Ifenprodil, Lubeluzole, Naloxone, Nalmefenem, Citicoline, Fosphenyloin, Lubeluzole, 619C89, BMS-204352).

19. A lymph-like composition for protecting the central nervous system of a mammal, as claimed in claim 1, wherein said Polypeptides comprising mainly chemically-linked amino acids with molecular weight between 5,000 to 30,000 Daltons.
